(19) United States
(12) Patent Application Publication
(10) Pub. No.: US 2005/0058718 A1
(43) Pub. Date: Mar. 17, 2005

(54) Preparations for cigarette toxicity reduction, health enhancement, and addiction elimination

(76) Inventors: Dehou Fei, New York, NY (US); MingYan Hu, New York, NY (US); Faye Zhengxing, Washington, DC (US); Gina Fei, New York, NY (US); Jolly Xing Zhou, New York, NY (US)

Correspondence Address: Dehou Fei;Apt. 2H, 3736 10th Ave., New York, NY, 10034, US

(21) Appl. No.: 10/681,103

(22) Filed: Oct. 9, 2003

(30) Foreign Application Priority Data
Sep. 17, 2003 (CN) 03151012.4

(51) Int. Cl. A61K 33/40; A61K 33/04; A61K 31/724

(52) U.S. Cl. 424/616; 424/702; 514/58

(57) ABSTRACT

This invention is a preparation with comprehensive and multi-functions—for reducing the toxicity in cigarette smoke, strengthening smokers' health and eliminating their addiction to smoking. More specifically, the liquid additive is designed to lower the toxin level in tobacco smoke on a broader scale and by a larger margin; the health pills, with their unfailing nutritious effect guaranteed, can fundamentally prevent smokers from getting various smoking related diseases; both the liquid additive and the health pills can also resolve and inactivate the addiction-causing substances in the human brain so as to help smokers quit smoking.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This invention is grounded in biochemistry, medicine, pharmacy, nutriology and other related fields

Presently, about 28 percent of men and 24 percent of women are smoking in the United States. Twenty-five percent of pregnant women who smoke quit during pregnancy; yet 80 percent resume smoking after childbirth. Tobacco smoking is responsible for 1 of every 5 deaths and is the most common cause of cancer-related deaths in this country (1).

Internationally, about 1.1 billion people smoke. Smoking is especially prevalent in developing countries and is continuing to increase. The health consequences of this addiction are severe. Children smoke 1.1 billion packs of cigarettes yearly, which accounts for more than $200 billion in future health care costs (1).

Over the past 30 years, the health communities at home and abroad have been working continuously to reduce the toxicity of tobacco smoke and assist smoking cessation. Some inventions did come out, but the effects were not comparable with the widespread harm of smoking. Our thorough searches throughout the world have shed light on the following issues: 1) Many inventions were limited to only reducing the toxicity of one or a few harmful chemicals. They provide no good solutions to the pressing problems caused by smoking; 2) some partially or completely depending on plants or herbs, even if effective, are not stable in their properties or have no secured sources of raw material for mass production and marketing. Recently the European Union drafted a bill to strictly control the import of plants. It will strike a heavy blow to those inventions when the law becomes effective not only in Europe but also in other countries; 3) some popular nicotine replacement products such as patches, chewing pieces, nasal sprays, inhalers and tobacco-infused drink are dangerous, because they only increase the intake of nicotine, a major causative factor of cancer, cardiovascular disease, respiratory illnesses and other diseases. As we know, nicotine releases a great deal of dopamine and glutamic acid in the brain. It is these two substances that caused addiction (including addiction to drugs). No wonder the mass media questioned the effect of using nicotine replacement products; 4) another challenge is an opinion in the legal battle against the tobacco industry that some “light” or “safe” cigarettes are deceptive, because people smoking such brands are likely to inhale more deeply and smoke more cigarettes to satiate their nicotine fix (2). This point of view only illustrates that toxicity reduction must be supported by gradual elimination of addiction to nicotine.

In view of the situation stated above and in answer to the call on banning tobacco from the World Health Organization, we are applying for a patent for our invention with the confidence that our additive will satisfy the health community, smokers, the tobacco industry, the government, or the whole society, because of its comprehensive multi-functions—reducing the toxicity in cigarettes, enhancing the health of smokers, and eventually eliminating the addiction. This is a scientific, safe, and effective invention based on our over 20 years of studies and experiments in medical science, biochemistry and other related subjects.

Over the past 20 years, we have studied a great deal of information in related fields through various books, journals, and internet. We especially referred ourselves to the sources in the “References” attached to following Detailed Description of the Invention.

### BRIEF SUMMARY OF THE INVENTION

This invention consists of two parts—a liquid additive and a supplementary health pill for tobacco industry and health communities to solve smoking related physical, social and psychological problems. This invention, proved unprecedented by our thorough search throughout the world, performs three major functions—toxicity reduction, health enhancement, and addiction elimination. The ingredients of the liquid additive and health pills are all within the scope of FDA's “Conventional Foods and Dietary Supplements.”

The liquid additive put into the cut tobacco in the process of cigarette production to reduce the toxins in cigarette smoke is called “ACA-104-1 and the health pills enclosed in the cigarette pack to prevent smokers from getting almost all smoking related diseases are called “ACA104-2A and ACA104-2B”. For a pharmaceutical reason, those ingredients designed for smoke cessation are conveniently included in either ACA-104-1 or ACA-104-2A/B.

The major advantages of this invention are as follows:

1. The liquid additive will effectively reduce or dissolve the toxins in more chemicals in tobacco smoke and by a bigger margin:
Among the 4000-odd compounds in tobacco smoke, forty are harmful to the human body. The seven most hazardous chemicals, however, are tar, nicotine (including its derivatives), carbon monoxide, nitric oxide compound, cyanhydric acid, radioactive material, and heavy metals such as cadmium, mercury, and arsenic.¹ For instance, our liquid additive will reduce the toxicity in tar by 75%-85%, 25% more reduction than that in a well-known brand of light cigarettes; some ingredients in the health pill will dissolve almost completely the carcinogenic polyene hydrocarbon epoxides left in the rest of tar. This liquid additive will also reduce nicotine by 90% to 97% and change it into nicotinic acid (VB3). This is a great creative step with respect to the toxicity reduction in cigarettes. Moreover, our health pill will also dissolve the toxicity in nitrosamine, a potent carcinogen before or after it is formed in the human body.
   ¹See Imperial Cancer Research Fund web site: “The Safer Cigarettes: what the Tobacco Industry Could Do. and Why It hasn't done It,” Mar. 3, 1999.

2. Our health pill will effectively prevent almost all kinds of smoking related diseases:
In selecting the ingredients for the health pill, we referred ourselves to a great deal of data drawn from all kinds of experiments, studies or clinic researches, as well as to our own experiences. Under the guidance of the theory of free radical, we based our selections on the effect of these ingredients on human cells and genes, on human nutrition levels, on strengthening the immune system, and on related clinic results. To ensure the unfailing effect of the health pill, we creatively adopted a co-enzyme to revitalize those oxidized ingredients and resume their important activity in preventing diseases.
In packaging, we enclosed the health pills in the cigarette pack for consumers to take in the anti-smoking ingredients in the right time and right dosage.

3. Our invention will eventually facilitate smoking cessation.
Since nicotine releases a great deal of dopamine and glutamic acid in the brain to cause smokers' addiction, our invention includes a set of effective ways to appropriately catabolize and inactivate dopamine and glutamic acid through transformation of enzymes and other biochemical changes.

4. Other advantages of this invention:
a) guaranteed sources of raw materials; b) cost of production is relatively low and thus competitive; c) no need to change the major equipment of cigarette production; d) though there will be a short-term training for operators and a workshop for making health pills, the process of sprinkling liquid additive to the tobacco is simple.

### BRIEF DESCRIPTION OF THE SEVERAL VIEWS OF THE DRAWING

Not Applicable.

### DETAILED DESCRIPTION OF THE INVENTION

Our invention consists of two parts: 1) a liquid additive spread into the cut tobacco in the process of cigarette production to reduce the toxicity (the so-called “ACA-104-1”) in tobacco smoke; 2) a supplemental health pills enclosed in the cigarette pack to prevent smokers from getting smoking related diseases (the so-called “ACA104-2A and ACA104-2B”). These pills will timely provide smokers with sufficient nutrition to reduce the toxicity in cigarette smoke and enhance their health. For a pharmaceutical reason, those ingredients designed to remove the addiction in smokers are conveniently included in either ACA-104-1 or ACA-104-2A/B. The liquid additive and supplemental health pill are interrelated and work in coordination.

1. Liquid Additive to Reduce Toxicity in Tobacco Smoke:

Among the 4000-odd compounds in tobacco smoke, over forty are harmful to the human body. The seven most hazardous chemicals, however, are tar, nicotine (including its derivatives), carbon monoxide, nitric oxide compound, cyanhydric acid, radioactive material, cadmium, mercury, and arsenic. They are regarded as the causes of smoking related diseases such as various cancers, cardiovascular disease, respiratory diseases, as well as other health problems—premature aging, weak immunity, tobacco amblyopia, mouth leukoderma, as well as other diseases caused by harmful radioactive material in tobacco smoke.

Is reducing toxicity in tobacco smoke necessary? Some people believe that a “low tar” read-out on the Federal Trade Commission's automated testing machine does not mean the smoker may inhale less harm from the “light” or “safe” cigarettes, because the smoker will draw harder to satisfy his/her crave for nicotine. Some experts, however, believe that reducing the toxicity in tobacco smoke is still a necessary first step since smoking cessation is not a task to be accomplished overnight. It must go through a transitional period, during which scientific inventions, social education, governmental regulation should all work together. American Medicine Association (AMA) once called for removing nicotine from tobacco within 5 to 10 years. AMA pointed out that since tobacco industry knows how to remove nicotine from tobacco as how to remove caffeine from coffee, the industry only needs to decide if nicotine should be removed all at once or gradually (3).

The all-or-nothing attitude toward reducing nicotine in cigarettes is not realistic if we admit that smokers need a transitional period to gradually get rid of his/her addiction. We therefore believe that the willpower to quit smoking should be supported by an invention to diminish and eventually break the addiction in the smoker's brain in the transitional period (We'll discuss this function of our invention later). Reasoning along this line, we hold that reducing toxicity including nicotine in tobacco smoke is a necessary first step.

We started detoxicating the major seven chemicals in tobacco smoke mainly through full oxidization. We found it easy to oxidize tar, carbon monoxide and nicotine effectively. It is also a cost-effective way to oxidize nicotine and transforming it into one type of vitamin B (nicotinic acid). With sufficient oxygen, tar will greatly lower its content, and the cancer-causing polycyclic aromatic hydrocarbons represented by 3, 4 benzo(a)phrene in tar will also decrease. The rest of polycyclic aromatic hydrocarbons in tar will lose its carcinogenicity when it combines with our anti-oxidants in our health pills; carbon monoxide, after oxidization, also loses its toxicity after it is changed into carbon dioxide.

To deal with nitric oxide compound, we used rare earth cerium dioxide as catalyst to replace precious metal platinum, thus separating nitrogen and oxygen from nitric oxide compound (5). Not only is it effective, it costs only one several thousandth of that of platinum; we detoxicated hydrocyanic acid by combining it with cysteine contained in the selenium-GSH-PX in our health pill, leaving the dissolved resultant to be discharged through human urine or slayer; those hazardous metals such as cadmium, mercury and arsenic were counteracted or inactivated by the selenium dioxide in our liquid additive (6); we applied the selenium-GSH-PX, the mainstay of our anti-oxidant in the health pill, to prevent the radioactive material 210 polonium (often found in tobacco) from triggering a lipidic peroxide reaction through free radicals. As a result, 210 polonium was detoxicated.

Not only do our liquid additive and health pill reduce or inactivate the toxicity in the seven major hazardous chemicals in tobacco smoke, they also transform some of the hazardous chemicals into healthful vitamins. Similarly, our liquid additive and health pill work effectively on carcinogenic compounds such as nitrosamine and ammonia in tobacco smoke. Ammonia, after counteracted, will lose its chemical irritation. Even after some ammonia enters the human body and raises the level of ammonia in blood, the VB6 in our health pill will combine the ammonia with γ-aminobutyric acid transformed from decarboxylated glutamic acid and the resultant urea will be discharged out of the human body (7); nitrosamine can be inactivated by the anti-oxidants in the health pill before or after nitrosamine forms in the human body. For the preparation for liquid additive, see Appendix 1.

Eleven years ago, we started the experiments on toxicity reduction. The Test Report by Shanghai Institute for Toxicity Control of Chemical Products proves that we reduced tar by 61.02% and nicotine by 81.18%. See Appendix 1, 2. With oxidants compound, catalysts, and full oxidization when necessary, we currently reduced nicotine by 90-97 percent and turned it into nicotinic acid (VB3); we reduced tar by 75 to 85 percent. Compared with some well-known “light” cigarettes, our cigarettes had 50 percent less nicotine and 25 percent less tar in tobacco smoke.
2. Health Pill to Enhance Smokers' Health and Prevent Diseases:

The components in tobacco smoke are very complicated and complex. They can invade all kinds of human systems and organs and cause various diseases. The most threatening and common are cancers, cardiovascular diseases and respiratory diseases. How to prevent these diseases effectively is our unprecedented challenge. The invention of our health pill was inspired by the theory of free radical, which states that free radicals are responsible for aging and degeneration of the human as well as other species (9). According to this theory, free radicals are the biochemical basis for many hazardous diseases such as cancers, cardiovascular disease, atherosclerosis, central nervous system, arthritis, muscle atrophy, and congenital malformation. With oxygen, free radicals' initiator will oxidize many biochemical elements inside and outside human cells. For instance, when the unsaturated fatty acid on the cell membrane is oxidized, which is called “lipidic peroxide reaction,” and if this kind of abnormal biological reaction is very strong, the structure and functions of human cells and cell membrane will be impaired, causing devious biochemical changes in DNA, RNA and enzymes. As damages vary in different systems, metabolic processes, and links, all kinds of biochemical deviations and diseases may occur. Free Radical Theory thus led us to believe that we could prevent and treat diseases more effectively if we zero in on human cells or molecules—the sources of diseases—to repair damages.

Selenium, a mineral and the major substance of our health pill, can form glutathione peroxidase (GSH-PX) in the human body and GSH-PX blocks the lipidic peroxide reaction initiated by free radicals. This indicates the important functions of selenium compound (pure selenium almost cannot be absorbed) and GSH-PX in the process of life such as suppressing peroxidization, cleansing free radicals, resolving peroxide, and repairing damaged molecules and cells. In view of the mechanism stated above, we centered our anti-oxidant prescription on sodium selenite, aiming at repairing the damaged molecules and cells, removing free radicals, resolving peroxide—the most fundamental way to prevent and cure diseases. These ingredients, within the scope of United States Recommended Dietary Allowance (RDA), will protect the structure and functions of cells and cell membranes to keep smokers in good health (10).

Since selenium is a necessary component of GSH-PX, its content is positively related to the activity of GSH-PX. So is the content of glutathione to the activity of GSH-PX. In the process of reducing the toxic peroxide and turning it into harmless hydroxy-compound, GSH-PX changes itself from a reductive GSH to an oxidative GSSG. At the same time, GSH-PX also resolves hydrogen peroxide in the human body, protecting the structure and functions of cell membranes from the impairment and interference by peroxide. Through the same GSH-PX, selenium can arrest the lipidic peroxide reaction caused by free radicals. All these functions to inhibit and prevent free radicals and their reactions are performed by either enzyme anti-oxidant or non-enzyme anti-oxidant in the human body. Diseases occur only when the defensive system is weakened by aging or unhealthy behaviors.

a. Selenium and Cancers:

Schrauzer reports that the data from the blood banks in 22 countries indicate that the selenium content in blood is in inverse proportion to the mortality rate of cancer patients (11); related data from 19 states of U.S. offered similar findings; a report measuring the selenium intake from diet in 29 countries also found a significant inverse relationship between selenium intake and the mortality rate of the people who had suffered from colon cancer, prostate cancer, breast cancer, ovary cancer, lung cancer, and leukaemia. This report therefore predicts that if the intake of selenium is doubled, the mortality rate of cancer patients in the United States and other Western countries will remarkably decrease (11); there is an obvious disparity between the average selenium content (0.49±0.22 ug/g) in the tobacco grown in the area with high selenium soil and that (0.16±0.05 ug/g) in the tobacco in the area with low selenium soil. While the former area had low tumor incidence, the latter presented high tumor incidence (11).

Various studies over the past 30 years also report that selenium, to some degree, checks all kinds of chemical causing cancers (including nitrosamine causing alimental canal cancer). When 3 ppm selenium was added to the drinking water for animals, it was effective on the leukoderma in their mouths and throats to the extent that it took two more weeks for cancers to form in the animals drinking water with selenium than that in the control group drinking water with no selenium. Two-week difference is statistically significant (12); other experiments also provide findings that selenium apparently inhibits the growth of spontaneous tumors, planted tumors, and tumors initiated by chemicals in different animals (12, 13).

In addition, selenium can prevent the growth of cancer-causing fungus and lower the toxicity of aflatoxin (12, p.8). A Japanese expert at Japan's Center for Cancer Studies believes that 60 percent of primary liver cancer incidences were caused by smoking and a smoker's liver starts to age 15 years earlier than that of a non-smoker; among lung cancer patients 72 percent are smokers; a smoker consuming 30 cigarettes daily is three times more likely to get lung cancer than a non-smoker (15).

After over 10 years of research and experiment, Professor Yu Shuyu at the Tumor Studies Institute of China's Medical Science Academy put forth first in the world that sodium selenite affects cell's genetic express, recording system, energy metabolism, and the structure of cell membrane. He points out that sodium selenite will reverse the proliferation, division and vicious exterior of tumor cells. After treated by selenium, the cancer cells external of the human body will decrease its carcinogenicity by 60 percent. His view is different from most researchers' opinion that to treat cancer is only to remove the oxidants and protect cells in the human body (16). Apparently Professor Yu has created a new vision in this field.

Selenium also inhibits the cancers caused by virus. For instance, if rats drank the water with 2 ppm and 6 ppm selenium, their incidence of rat's breast cancer caused by external highly carcinogenic rat's breast cancer-causing virus will decrease by 50 to 80 percent. The tumors on the rats in the experimental group grew more slowly and were less vicious than that of the control group. The rats in the experimental group also lived longer than their counterparts in the control group (12).

Some experiments demonstrate that even if selenium was provided outside of a rat's intestines, it could still check the growth of cancer cells planted in the rat. Experiments on cancer-causing chemicals report that if rats eat the food with 1-2 ppm selenium, their cancer incidence will go down by 30 to 40 percent; little rats drinking the water with 1-2 ppm sodium selenite, their tumor incidence will decrease by 70 percent (12).

The findings from “Thirty-Eight Cases on Selenium Content in the Hair of Leukaemia Patients”: the selenium content in the hair of the control group (healthy people) is 0.762±0.279; the selenium content in the hair of acute leukaemia patients is 0.515±0.216, P<0.001. This disparity is significant. It shows that these patients had long been in the state of low selenium. This finding is also consistent with the clinic tests by Yu Daowen and others, which indicate that the selenium contents in the marrow and blood of leukaemia patients are lower than that of the control group. Their findings further confirmed other reports that selenium can cure and check cancers (including leukaemia). The theory behind the findings is that when selenium content is insufficient, the human body tends to be more susceptive to cancer-causing factors (17).

More studies indicate that selenium will stimulate the growth of immunoglobulin and antibody and thus strengthens the human immune system against diseases. An experiment proves that the immunoglobulin content in the little rats fed with selenium is 20 to 30 times more than that of the control group fed no selenium (21).

An epidemiologist at the Arizona Cancer Center and a selenium scholar Larry Clark tested the anti-cancer effect of selenium additive in human body. Their clinic reports state that they collected statistics on 1300 people who took 200 ug selenium daily for five years. Their incidence of lung cancer was reduced by 46 percent, large intestine and rectum by 58 percent, prostate cancer by 63 percent. Even if a selenium taker is sick from cancer, he or she has 50 percent more chance to survive (18).

The experiments above and the related data all illustrate the functions of selenium to inhibit cancers caused by chemicals, virus, fungus and other hazardous elements, its effect on the gene and the immune system, as well as the results of applying selenium and GSH-PX to clinic trials. Our studies further prove that with our extra adjuvant, the effect of the health pill will multiply.

b. Selenium-Antioxidant and Cardiovascular Disease:

The close correlations between selenium and the structure, functions and causes of cardiovascular disease have drawn the attention of the world. In the United States, Finland and other countries the incidences of coronary heart disease and hypertension in the area with high selenium are both lower than that in the area with low selenium (19). The same pattern was shown in the incidences of brain thrombus, rheumatic heart disease, chronic endocarditis, and the whole body arteriosclerosis. A report from Finland also proves that the selenium content in the blood and blood serum of myocardial impaction patients are lower than that of healthy people; selenium is effective in treatment of cardiovascular disease (20).

Vleet points out that selenium and vitamin E are important to the structures and functions of myocardial fibrous, arterioles and capillaries of many animals (30). Huang Bixia and others report that selenium content affects the activity of the enzyme in the microsomes of the liver. It therefore directly affects the catabolism of cholesterol in the human body. Lack of selenium will decrease HDL-C, thus lowering the ratio of HDL-C to the total quantity of cholesterol. Furthermore, lack of selenium will decrease the activity of HDL-C and thus increase the content of lipidic peroxide. These are dangerous factors causing coronary atherosclerosis; if cerebral embolism occurs, the damage on nerve cells is related to free radicals and to the form of lipidic peroxide. Normally brain thrombus patients have lower level of selenium (25). Some Finnish medical experts found that taking the anti-oxidant vitamin C and vitamin E for at least three years will effectively prevent arteriosclerosis, especially the cervical vertebra arteriosclerosis, an important factor of stroke, for male smokers. Their experiments indicate that the arteriosclerosis level of those patients who took vitamin E and vitamin C is only 45 percent of that of those who did not; three quarters of men who took vitamin E and vitamin C eased their level of arteriosclerosis (29).

The studies by Dr. John Ots prove: A smoker's thrombocyte is more likely to coagulate, thus prompting the incidence of heart attack and stroke. This is because smoker's thrombocyte tends to discharge a sticky material that will block arteries. He also believes that VB6 affects the catabolism of fat. Lack of VB6 will cause atherosclerosis. The reason for VB6 to prevent thrombus is related to its special combination with the albumen on the surface of thrombocyte, its participation in the transformation with sugar, amino acid and catabolized enzyme, and its function to prevent thrombocyte from coagulating (26). The circle of medical science has generally recognized the contribution made by nicotinic acid to cardiovascular disease and has made it, with vitamin C or inositol, a patent medicine on the market.

c. Selenium, Other Anti-Oxidants and Respiratory Diseases:

Selenium and other anti-oxidants also have excellent effect on respiratory diseases. An expert at Connell University and a Chinese scholar point out that a diet rich in anti-oxidants will strengthen lung functions and prevent respiratory diseases such as asthma, pulmonary emphysema and chronic bronchitis. The effect of these anti-oxidants on the lung has a lot to do with smoking. The experts also state that taking in B-carotin with selenium and high dosage anti-oxidants is a very good way to prevent cells from damages by hazardous biochemical elements (28). A medical study took 18,162 adults as their samples to examine the relationship between the ingredients (we adopted in our health pill) of their diet and their blood and their lung functions in a period of six years. The experts found strong evidence to prove, through comparing the variables such as gender, age, bodily fat, race, income, and smoking or non-smoking behavior, that these ingredients are closely related to these adults' lung functions. Their findings also indicate that those natural ingredients in food are not as effective on smokers' lung functions as that of their chemical counterparts (such as vitmin C and E) on both smokers and non-smokers (28).

d) Selenium and Other Smoking Related Diseases:

In addition, this health pill also prevents other smoking related diseases such as premature aging, weak immunity, tobacco amblyopia, mouth leukoderma and other diseases caused by harmful radioactive material in tobacco smoke.

e) Our Creative Invention:

Over the past 30 years selenium and its compound GSH-PX have displayed wonderful performances in animal and clinic experiments in various countries, but the activity of GSH-PX decreases because of oxidization other factors. Even with the supplement of selenium, GSH-PX will not resume its activity due to a series of chemical reactions such as oxidization, replacement, resolve, and counteraction. GSH especially loses its function as an anti-oxidant after it is changed into an oxidant GSSG.

We tackled the essential problem: how to reduce GSSG back to GSH. We believe we could not just mix more GSH with glutamic acid, systeine and glycine. Instead, we must find a synthetase and reduce the oxidized GSSG in the human body. GSSG is an oxidized enzyme protein, a complicated complex. Though we knew there is GSSG-R (GSSG's reductase) in the human body, it is not active enough. We knew we were in need of a strong co-enzyme as catalyst to reduce GSSG back to GSH.

After a long search and study, we finally found a co-enzyme to reduce GSSG back to GSH. We were greatly encouraged by this finding. With this co-enzyme, we could resume the activity of GSH in every way. Moreover, in case GSSG-R is insufficient, the two components in riboflavin in the health pill—flavin mononucleotide (FMN) and flavin adenine dinucleotide (FAD)—will resume the activity of GSSG-R to the normal level. We believe this is a breakthrough in generating an unfailing effect to prevent smokers from getting various smoking related diseases. For the preparation for the health pills, see Appendix 3.

3. The Causes of Addiction and Our Invention in Smoking Cessation:

It is a consensus of medical experts that when nicotine enters the human brain, brain cells will release a great deal of dopamine and glutamic acid, the major causes of addiction to cigarettes (or addiction to other drugs) (22, 23, 24). An expert from Virginia Institute of Technology reports that they have separated a compound from tobacco, which will inhibit monoamine oxidase (MAO), a major enzyme in the brain to resolve dopamine (27); another series of research papers from a state lab in New York City indicate that the MAO concentration in a smoker's brain is 40 percent lower than that of a non-smoker. This is the reason why a smoker has higher dopamine content in the brain than a non-smoker (27). Some researchers from Columbia University state in “Science” magazine that when nicotine enters the brain, it greatly increases the release of glutamic acid in the brain. They detailed the reasons why it is an accomplice in causing nicotine addiction (24).

The United States and Canada are doing clinic experiments on γ-VINYL GABA, GVG, a medicine produced by Aventis in Europe for children's epilepsy, to see if it could be used to detoxicate tobacco smoke and facilitate smoking cessation. GVG may reduce the dopamine in the smoker's brain and thus help him/her break the addiction to nicotine. We believe, however, that it will not be enough to only reduce dopamine in the brain, because glutamic acid, through decreasing MAO's activity, makes it harder to resolve and inactivate dopamine. Glutamic acid might be one of the causes that stabilizes the addiction to nicotine and other drugs. Furthermore, glutamic acid will also damage brain cells and cause the death of brain cells. In addition, iron, if over taken or stored in the human body, will also release glutamic acid in the brain. Excessive iron may be stored in the body for many reasons: Some people tend to supplement too much iron to cure anemia. Some people cook in pots or pans made of iron, drink too much beer or grape wine, or who have iron residue caused by excessive blood transfusions, or indigestion by intestine. Furthermore, there are other factors causing the release of glutamic acid. For instance, lack of vitamin B6 and nicotinic acid will increase the pathologic absorption of iron; lack of vitamin E and other anti-oxidants will cause iron chronic poisoning. Not only will these factors cause more glutamic acid in the brain, they will also cause other diseases. To eliminate the addiction to nicotine, therefore, we must appropriately reduce dopamine, glutamic acid, and at the same time reduce the excessive iron in the human body. We adopted the following three methods:

(a) Strengthening MAO's activity with Copper Compound and Enhancing Copper's function with Manganese Compound:

Since glutamic acid lowers the copper content in the brain and copper is an important component of MAO, glutamic acid, therefore, may decrease the activity of MAO. [Kong Xiangrui reports that glutamic acid injection will lower the copper content in human blood serum (6. p. 173)].

To supplement copper and manganese compound, however, is not easy, because they are metal oxidants, which may counteract with the majority of the anti-oxidants in the health pill. Furthermore, several hours after copper compound enters the human body, it will compound into ceruloplasmin in the liver. This ceruloplasmin, however, cannot go through the blood-brain barrier to increase copper. Later we came up with the idea of adding copper and manganese into the cut tobacco. When the smoker lights up, he/she will absorb the metals through his/her respiratory tract. Thanks to the minim quantity of the copper and manganese as catalysts, there is no problem in safety. Our design is based on the knowledge that dopamine could go through two paths of catabolism, but both need the decomposition of MAO and the ultimate outcomes of both is the same organic acid. We thus had a good grasp of the chemical formulas of their catabolism.

(b) Use Vitamin B6 (changed into pyridoxal 5-phosphate in the human body) as the Co-Enzyme for Glutamic Acid's Decarboxylation:

The chemical formulas to decarboxylate glutamic acid and change it into γ-aminobutyric acid (GABA) are as follows: 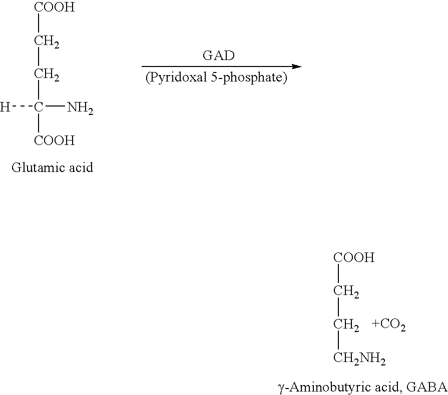
The distribution of GAD in the nervous system parallels with the content of GABA. In other words, where GAD is active, so is GABA. With sufficient vitamin B6 taken in, the chemical reactions above will proceed smoothly. In this way, a dramatic situation thus occurs: The negative functions caused by glutamic acid—damaging brain cells to death, decreasing the activity of MAO, stabilizing the addiction to nicotine or drugs—will be changed into positive functions such as protecting brain cells and resuming their functions, sobering the patients from the faint caused by carbon monoxide or uremia. The main positive function, however, is the decrease of addiction. In addition, GABA also will combine with ammonia in the blood caused by smoking to form urea to be discharged out of the human body.
(c) Control the Iron Taken-In and Stored:

Since excessive iron will release glutamic acid in the human brain, we also had to control the iron content in the human body. Our healthy pill includes some ingredients to keep iron along its regular path of catabolism and prevent excessive iron from being stored in the human body; VA, VE, VC and a bit of copper and manganese compound will all turn iron into another type of iron easy to be catabolized and discharged out of the human body while the nicotinic acid and VB6 in the health pill prevents pathological absorption of excessive iron.

### REFERENCES

(1) Sat Sharma, “Nicotine Addiction,” University of Manitoba, www. Emedicine.com/med/topic 1642.htm#section˜bibiliography, Jan. 21, 2003.
(2) Eric Lichtblau, “U.S. Seeks $289 Billion in Cigarette Makers' Profits,” Washington Post, Mar. 17, 2003.
(3) “AMA Calling for Eliminating Nicotine from Tobacco,” World Daily, Jun. 19, 1998.
(4) Xie Huimin, et al. “Knowledge on Vitamines,” People's Health Publishing House, 1985, p.98.
(5) Intelligence Institute of Shanghai Chemical Industry, “Rare Earth Applied to Catalyst,” 1982, p.7-9.
(6) Kong Xiangrui, “Essential Trace Element: Their Nutrition, Physiological and Clinic Significance,” Anhui Science & Technology Publishing Society, 1982. p.296; p.300-301; p.173.
(7) Zhou Ziyong, et al. “A New Handbook on Common Medicines,” Gold Shield Publishing House, 1995, p. 274.
(8) Shanghai First Academy of Medical Science, et al. “Biochemistry Applied to Medicine,” First Vol, 1979, p.757.
(9) An Interview with Dr. Denham Harman—Father of the Free Radical Concept of Disease, Life Extension Magazine January 1998 at http://www.lef.org/magazine/mag98/jan-interview98.html
(10) Armstrong, Clare “Recommended Dietary Allowance (RDA),” National Academy of Sciences; 10^{th} ed., 1989.
(11) Bogen J. D. etal: JNCL, 66:27, 1981.
(12) Xue Junwu, “Foreign Medical Science,” Edition on Ear, Nose and Throat, Vol. 1, 1987. p. 7, 8.
(13) Jocobs M, et al: “Inhibitory Efforts of Selenium on 1,2-Dimethyl-hydrazing and Methlazoxymthanol Acetate Industry of Colon Tumors, Cancer Hett,” 2:133, 1977.
(14) Ip C.Factos: “Influencing the Anticarcinogenic Efficacy of Selenium in Dimethyl-Benz (a) Anthracene-Induced Mammry Tumorigensis in Rats, Cancer Res,” 41:2683, 1981.
(15) Shanghai Broadcast Station: “Hygiene and Health,” Jan. 3, 1990.
(16) Yu Shuyu, “Trace Element,” 1989, Vol. 3, p. 27.
(17) Yu Daowen, “Trace Element,” Vol. 4, 1988. p. 43.
(18) Larry Clark, “Selenium Reduces the Danger of Cancer,” Journal of American Medical Association. Dec. 5, 1996.
(19) Robinson M F: Am J Clin Nutr. 32:1477, 1979.
(20) Frost D V, et al: “Ann Rev Pharmacal,” 15:259, 1976.
(21) George E. Berkley, “Cancer: How to Prevent It & How to Help Your Doctor Fight It,” translated by Chen Zuhui, et al; Prentice-Hall, Inc., 1978. p. 65.
(22) Overseas Chinese, “The Substance Inducing Brain to Take Drugs,” May 29, 1999.
(23) A Paper by Brooklyn State Lab republished by Xinming Evening Post, Mar. 4, 1996, p.5.
(24) Chen Zonglun, “Uncover the Myth of Nicotine Addiction,” Translated News in Science and Technology, Oct. 25, 1995.
(25) Huang Bixia, “Trace Element,” Jan, 1989, p.18.
(26) Wu Qiushen, Xinming Evening Post, Aug. 17, 1988 (6).
(27) U.S.-Sino Medical Science Website @aol.com, “Tobacco and Chinese Medicine: A Compound against Parkinson's Disease,” April, 2000.
(28) “Anti-Oxidant Diet Strengthens Lung Functions,” World Daily, Apr. 19, 1998, A 1.
(29) U.S.-Sin θ Medical Science Website @aol.com, “Vitamins Compound Prevents Arteriosclerosis,” Feb. 6, 2001.
(30) Fleet J F et al, Lab Invest, 37: 201. 1977.

### Appendix 1

PRESCRIPTION, PREPARATION & USE
OF THE LIQUID ADDITIVE TO TOBACCO
(ACA-104-1)

| | | Quantity (3) | | |
|---|---|---|---|---|
| | | Content | | |
| | Components & | Range % | | |
| Order of | Concentration | (Per 100 g | | |
| Sprinkle | of Ingredients | Tobacco) | | Preparation |
| | Tween-80 | 0.7-15 | ml | Dissolve them for use later; label the |
| | Hot water | 50-100 | ml | solution as A. |
| | (55-60° C.) | | | |
| (1) | Cerium dioxide | 7-134 | mg | Dilute sulfuric acid to 20% solution, take |
| | Sulfuric acid | 0.2-10 | ml | out 6.665 ml solution (H₂SO₄1.333 ml), into |
| | (5%-20%) V/V | | | which put 44.45 mg of CeO₂, stir them for |
| | | | | use later; label this solution as B; |
| | Selenium | 0.4-8 | mg | Dissolve 2 mg selenium dioxide into 1 ml |
| | dioxide | | | water; |
| | B-cyclodextrin | 0.1-4 | g | Put 1.85 g B-Cyd into 100 ml water at 50-60° C.; |
| | | | | Take out 12.973 ml (which equals B- |
| | | | | Cyd 0.24 g) to mix the SeO₂ sol. above; stir |
| | | | | till all dissolved; label it as Solution C; |
| | | | | Put the A, B, C three solutions above |
| | | | | together, stir them thoroughly; sprinkle |
| | | | | them onto 100 g tobacco; blend them |
| | | | | thoroughly and put it aside for 30 minutes; |
| (2) | Hydrogen | 30-600 | ml | Sprinkle 3% Hydrogen peroxide 167 ml |
| | peroxide (3% or | | | onto 100 g tobacco above, blend them |
| | 6%) | | | thoroughly and stir it for 30 minutes; |
| | *Potassium | 15-150 | mg | Dissolve KMnO₄ to 1% solution; take out |
| | permanganate | | | 4.445 ml (which equals 44.45 mg KMnO₄); |
| | | | | Dilute Sulfuric acid to 5% solution; take out |
| | | | | 6.66 ml (which equals 0.333 ml H₂SO₄); |
| | | | | label this solution as D; |
| (3) | *Cupric Sulfate | 45-400 | mg | Dilute CuSO₄ to 5% solution; take out |
| | | | | 2.666 ml (which equals 133.33 mg CuSO₄); |
| | | | | label this solution as E; |
| | | | | Mix D with E; and stir them thoroughly for |
| | | | | use later; |
| | Cupric Oxide | 20-270 | mg | Grind fine CuO (66.67 mg) and activated |
| | B-cyclo-dextrin | 0.1-4 | g | MnO₂ (33.335 mg); |
| | | | | Dissolve 1.333 g B-Cyd into 72.054 ml hot |
| | | | | water, into which put the above CuO and |
| | Activated | 10-100 | mg | MnO₂; grind (with motor grinder) and stir |
| | Manganese | | | them for an hour or more till it becomes a |
| | dioxide | | | thin paste; label it as F; |
| | | | | Mix D, E, F together and sprinkle it onto |
| | | | | the 100 g tobacco above; stir them. |
| (4) | 3% hydrogen | | | Directly sprinkle Hydrogen peroxide onto |
| | peroxide | | | the tobacco above; blend them thoroughly at |
| | | | | 40-45° C. for 2.5 hours; |
| | | | | Add the essence and seasoning condiments |
| | | | | for making cigarettes; stir it while baking |
| | | | | till it reaches the moisture required. |

Note:
1. After this liquid additive is sprinkled onto the tobacco and is thoroughly mixed with tobacco, the PH value of the tobacco juice should be 5 to 7.
2. The ingredients marked by * are also part of the prescription to eliminate the smoking addiction. The rest for this function is listed in the prescription of the health pill.
3. We assume each cigarette contains 0.75 g tobacco.

PRESCRIPTION, PREPARATION & USE
OF THE HEALTH PILL ENCLOSED IN CIGARETTE PACK
(ACA-104-2A, B)

| | | Quantity | | | |
|---|---|---|---|---|---|
| | | Content (per | | | |
| | | 100 g | | | |
| | Name of | tobacco) | | | Signature |
| Kind | Components | Range % | | Preparation | (Sig.) |
| | *Sodium selenite | 0.2-14.6 | mg | Dissolve Na₂SeO₃ | |
| | | | | (3.65 mg) in 1 ml water; | |
| | B-cyclodextrin | 0.5-30 | ml | Add 2 ml B-Cyd | Dose of orange |
| | (1.85%) | | | (1.85%) to the | pill: |
| | | | | above solution; | For those who |
| | | | | stir them | smoke 20 cigs |
| | | | | thoroughly; | daily, take 2 |
| | *Vitamin E | 0.2-10 | g | | pills before |
| (A) | *Vitamin A | 3-35 | mg | Grind them fine | eating in the |
| | Butylated | 0.3-27 | mg | | morning or half |
| | hydroxytoluene | | | | an hour before |
| | Riboflavin | 7-200 | mg | | smoking; Take |
| | Nicotinic acid | 7-2000 | mg | Mix them; | 2 pills one hour |
| | Pyridoxine | 33-2000 | mg | | after lunch; |
| | hydrochloride | | | | For those who |
| | | | | Mix all the above | smoke 10 cigs |
| | | | | thoroughly and | daily, take 1 pill |
| | | | | make four orange | twice a day at |
| | | | | coated pills. | the same time |
| | | | | | as stated above. |
| (B) | *Ascorbic acid | 0.07-6 | g | Make two white- | Dose of the |
| | | | | coated pills with | white pill: |
| | | | | 400 mg ascorbic | For those who |
| | | | | acid.. | smoke 20 cigs |
| | | | | | daily, take 2 |
| | | | | | pills one hour |
| | | | | | after the last cig |
| | | | | | before the |
| | | | | | bedtime. |
| | | | | | For those who |
| | | | | | smoke 10 cigs, |
| | | | | | take 1 pill at the |
| | | | | | time stated |
| | | | | | above. |

Note:
The ingredients marked by * are also part of the prescription to eliminate addiction. Other ingredients for this function are listed in the prescription of the liquid additive.

Contraindications:
1) Pregnant women are inhibited from taking these pills;
2) While taking these pills, the smoker is inhibited from drinking alcohol, because alcohol will decrease the effect of the health pill or cause some physical discomfort. The smoker may drink one cup of light alcohol three hours before or after taking the pills, but drinking alcohol is allowed no more than one time a day.
   Remarks: With riboflavin in the pill, it is normal for the consumer to find his/her urine green-yellowish.

1. A liquid additive and a supplementary health pill with comprehensive and multi-functions for making a new type of cigarette:
The functions of the additive and health pill include reducing toxicity in tobacco smoke, strengthening the health of smokers, and gradually eliminate the addiction to nicotine to quit smoking. These functions are interrelated and work in coordination.

2. Reducing toxicity on a broader scale and by a larger margin:
The liquid additive will reduce the toxicity in tobacco smoke such as nicotine, tar (including polycyclic aromatic hydrocarbons represented by 3, 4 benzo(a)phrene), carbon monoxide, nitric oxide compound, hydro cyanic acid, cadmium, mercury, arsenic, as well as the particular nitrosamine in tobacco; For instance, this additive will reduce nicotine by 90% to 97% (50% more reduction than “light” cigarettes), tar by 75% to 85% (25% more reduction than “light” cigarettes).
In light of the claim 2 above, we designed the following compositions and contents of the liquid additive for 100 grams of tobacco:

| Components | Content | |
|---|---|---|
| Tween-80 | 0.7-15 | ml |
| Hot water (55-60° C.) | 50-100 | ml |
| Cerium dioxide | 7-134 | mg |
| Sulfuric acid (5%-20%) V/V | 0.2-10 | ml |
| Selenium dioxide | 0.4-8 | mg |
| B-cyclodextrin | 0.1-4 | g |
| Hydrogen peroxide (3% or 6%) | 30-600 | ml |
| Potassium permanganate | 15-150 | mg |
| *Cupric Sulfate | 45-400 | mg |
| Cupric Oxide | 20-270 | mg |
| Activated Manganese dioxide | 10-100 | mg |

Note:
The components with * are those also for the function of eliminating smoking addiction.

3. Preventing more diseases and strengthening health more effectively:
This health pill is designed to repair the damages at a molecule or cell level, wipe out free radicals, and resolve peroxide in order to protect the structures and functions of cell and cell membrane. It can prevent a broad scope of diseases including various cancers, cardiovascular diseases, respiratory diseases, and other smoking related diseases and problems. We creatively apply a co-enzyme to resuming the activity of glutathione peroxidase (GSH-PX) so as to guarantee its unfailing effect in reducing tobacco toxicity and preventing diseases.
In light of the claim 3 above, we designed the following components and contents of the health pills for 100 gram of tobacco (about 20 cigarettes x 6.667 packs):

| Components | Contents | |
|---|---|---|
| Sodium selenite | 0.2-14.6 | mg |
| B-cyclodextrin (1.85%) | 3.5-67 | ml |
| *Vitamin E | 0.2-10 | g |
| *Vitamin A | 3-35 | mg |
| Butylated hydroxytoluene | 0.3-27 | mg |
| Riboflavin | 7-200 | mg |
| *Nicotinic acid | 7-2000 | mg |
| *Pyridoxine hydrochloride | 33-2000 | mg |

The components above are for coated pill ACA104-2A and the following is for coated pill ACA 104-2B:

| Components | Contents |
|---|---|
| *Ascorbic acid | 0.07-6 g |

Note:
The components with * are those also for the function of eliminating smoking addiction.
Dose: For those who smoke 20 cigs daily, take 2 pills before eating in the morning or half an hour before smoking; take 2 pills one hour after lunch.
For those who smoke 10 cigs daily, take 1 pill twice a day at the same time as stated above.

4. Eliminating addiction safely and effectively:
Since nicotine releases a great deal of dopamine and glutamic acid in the human brain, which are the major causes of addiction, we designed three channels to decrease dopamine and glutamic acid by using a) copper compound & manganese compound to strengthen MAO's activity; b) VB6 to turn glumatic acid to y-aminobutyric acid (GABA); c) VA, VE, VC and other anti-oxidants to prevent excessive iron in the body from releasing glutamic acid in the brain. It is a more scientific, effective and safer way in smoking cessation than using nicotine replacement products.
